(19) United States
(12) Patent Application Publication
(10) Pub. No.: US 2023/0218557 A1
(43) Pub. Date: Jul. 13, 2023

(54) A FATTY ACID BASED COMPOSITION FOR TREATMENT AND/OR PREVENTION OF ENVELOPED-VIRUS RELATED INFECTIONS

(71) Applicant: PALANI LLC, Chester Springs, PA (US)

(72) Inventors: ANUPAM DOKENIYA, Somerville, MA (US); KUMARIL BHARGAVA, Chester Springs, PA (US); DINO ROTONDO, Scotland (GB); LALIT AMBASTHA, New Delhi (IN)

(21) Appl. No.: 17/998,161

(22) PCT Filed: May 14, 2021
(86) PCT No.: PCT/IB2021/054161

§ 371 Date: Nov. 8, 2022

Related U.S. Application Data
(60) Provisional application No. 62/704,515, filed on May 14, 2020.

(51) Int. Cl. A61K 31/202; A61K 31/201; A01N 37/06; A01N 25/30; A61K 47/40; A61P 31/14; A01P 1/00

(52) U.S. Cl. CPC A61K 31/202; A61K 31/201; A01N 37/06; A01N 25/30; A61K 47/40; A61P 31/14; A01P 1/00

(57) ABSTRACT

A fatty acid based composition for treatment and/or prevention of enveloped-virus related infections by administering fatty acid based compositions. The present invention further provides a free fatty acid based sprayable composition to prevent and/or treatment of SARS-Virus related infections. The fatty acids used in the present invention are Linoleic acid (LA), α-linolenic acid (ALA) and gamma-linolenic acid (GLA). In a preferred embodiment, we propose that topical administration of naturally occurring fatty acids in an optimal formulation may be beneficial in the prophylaxis of respiratory enveloped viruses and in particular the lipid-rich SARS-CoV-2 virus. This could be administered via nasal and/or oral route. The composition comprises at least one fatty acid, a suitable carrier, preservative, cosolvents and surfactants and/or cyclodextrin used as an excipient that may also enhance anti-viral properties.

### FIELD OF THE INVENTION

The present invention relates to a fatty acid based composition for prevention and/or treatment of viral infections. More particularly, the present invention provides one or more fatty acid/acids based nasal and/or oral composition to prevent and/or treatment of Enveloped-Virus related infections.

### BACKGROUND OF THE INVENTION

The eruption of the novel coronavirus disease, COVID-19, caused by the novel coronavirus, 2019-nCoV, officially designated as severe acute respiratory syndrome coronavirus 2 (SARS-CoV-2), has been declared as world epidemic by WHO The infection by SARS-CoV-2 could be traced to zoonotic transmission with clinical symptoms of fever and cough which further leads to lower respiratory tract diseases. Presently, there is no specific antiviral drug available for this disease (Wu Z, McGoogan J M., 2020).

Coronaviruses are large, enveloped, plus-stranded RNA viruses with a helical nucleocapsid (Machhi J et al., September 2020). They cause common cold in all age groups accounting for approximately 15% of all colds. Corona viruses are a major cause of common colds in the winter months. They have been implicated in the etiology of gastrointestinal disease in infants. They also cause economically important diseases in animals (e.g. avian infectious bronchitis and porcine transmissible gastroenteritis).

Coronaviruses get their name because in electron micrographs the envelope glycoproteins appear to form a halo or corona around the periphery of the virion (Kahn J S & McIntosh K., 2005). The virus is found throughout the world. Antibodies begin to appear in childhood, and are found in more than 90% of adults. The frequency of coronavirus respiratory infections is highly variable from year to year. The highest incidence occurs in years when rhinovirus colds are lowest and coronavirus colds tend to occur in defined outbreaks.

Like other coronaviruses, SARS-CoV-2 are spherical in shape and have protein spikes on their surface that help the virus to attach to the human cells, which further undergoes structural change on fusion and facilitates the viral genes to enter the host cell. Interestingly, angiotensin converting enzyme 2 (ACE2) is the cellular receptor for SARS-CoV-2, which is expressed in the membranes of various cells in the body. SARS-CoV-2 spike binds ACE2 on human cells which enable SARS-CoV-2 to spread more easily from person to person (Wrapp D et al., 2020).

SARS-CoV-2 shares about 80% genome identity with that of SARS-CoV and about 96% similarity to BatCoV RaTG13 (Zhou P. et al., 2020). Despite similarities with SARS-CoV, three different antibodies against the 2002 SARS virus could not bind to the SARS-CoV-2 spike protein successfully. Therefore, there is a need of more specific treatment strategy for SARS-CoV-2. Presently, the treatment provided by healthcare practitioners is customized according to the symptoms and severity experienced by the patient. Currently, there are no existing licensed anti-viral therapies that are specific for COVID-19. Therefore, there is an urgent requirement for the development of such treatment.

Many viruses have a lipid-rich protein outer envelope coating which render them susceptible to detergent action. Indeed, detergents have been used to safely inactivate enveloped viruses in blood plasma to be used for transfusion without affecting the efficacy of the plasma for patient treatment (Hellstem & Solheim, 2011). The detergent treatment in these processes is generally carried out ex vivo since the use of detergents, is not easily transferable to internal human use as it is unlikely that epithelial surfaces in vivo would tolerate a continued exposure to strong detergents. However, this raises the possibility that other biocompatible and naturally occurring molecules with amphipathic properties may suppress or possibly inactivate enveloped viruses directly on epithelial surfaces.

In the current context, SARS-CoV-2, the virus that causes COVID-19, is a lipid-rich enveloped virus in which the lipid coating is not only critical to viral integrity but is also involved in its infectivity by facilitating endocytosis through host/target cell membranes (Baglivo et al., 2020). This virus also appears to survive extracellularly on a variety of surfaces but can be inactivated by detergent treatment (van Doremalen et al., 2020). AU619797 relates to antiviral activity of fatty acids and monoglycerides on enveloped viruses. AU2006223065B2 discloses topical antiviral compositions comprising of fatty acid ester, fatty ether or alkoxide which when applied to the mucosal tissues are useful in the treatment and/or prevention of conditions that are caused or aggravated by, microorganisms (including viruses). An early study (Kohn et al., 1980) showed that a wide variety of enveloped viruses including influenza, herpes and sendai viruses could be inactivated by long chain unsaturated fatty acids such as oleic and linoleic acids. This occurred within minutes with concentrations in the 5-25 μg/ml range (Kohn et al., 1980). Such concentrations should be tolerable by epithelial surfaces (>100 μM). A subsequent study confirmed the virucidal actions of long chain unsaturated fatty acid (Thormar et al., 1987, short chain or saturated variants had no effects). This study showed that inactivation occurred within 30 minutes at 37° C. The most effective fatty acids were linoleic acid and gamma-linolenic acid, which were shown to lyse and completely disintegrate viral particles as confirmed by electron microscopy.

A study in 2010 showed that the enveloped influenza virus strain H1N1 could be completely inactivated by household detergent (Greatorex et al., 2010). Fletcher et al., 2020, disclosed a novel formulation named ViroSAL, having caprylic acid as one of the main ingredients; that inhibits a range of enveloped viruses. They evaluated a formulation for its ability to inhibit a range of viral infections in vitro and in vivo.

The pandemic Covid-19 disease has become a serious threat to the global public health due to unavailability of effective drugs or vaccines to treat or prevent this infectious disease. Hence, novel drugs and new approaches to develop them are urgently needed. Both de novo drug discovery and drug repurposing have been used in the search for an effective antiviral drug. Unlike the lengthy and costly process of de novo drug discovery, drug repurposing can reduce the time, cost and risk associated with drug innovation.

There are available in the state of the art various drug formulations to provide symptomatic relief in viral infections. However, prophylactic approaches are very less where once the microbial contraction happens, spread of the same can be contained in body to avoid aggravation of symptoms. Another challenge posed is type of medicament. Medicaments taken by mouth and swallowed are absorbed first into the blood perfusing the gastrointestinal tract. The venous drainage from the GI tract is into the blood perfusing the liver. Certain areas of the alimentary canal have a venous drainage which does not involve a first pass through the liver. These areas (the mucous membrane of the buccal cavity, under the tongue and the nasopharynx, and the distal rectum) drain directly into the left side of the heart. The avoidance of the first pass effect is the rationale for the use of buccal, nasal and sublingual formations, and suppositories. Both sublingual and buccal formulations depend on the efficient transfer of medicament from a hydrophilic vehicle to the mucous membrane of the sublingual or buccal mucosae. Transfer of medicament through the interstices between or through epithelial cells is governed principally by the lipid solubility of the medicament. Where a drug is water insoluble this is a further barrier to absorption from the sublingual area. There are therefore physical and biological limitations on the therapeutic usefulness of lipophilic medicaments given by mouth and swallowed.

When it comes to oral medicaments, herbal infusions, lozenges and candies containing herbal extracts with soothing or numbing ingredients are often the first choice. However, ingredients in lozenges and candies are released quite slowly and are not always targeted within the oral cavity. It is thought that the main route of infection by SARS-CoV-2 is inhalation and subsequent adherence of viral particles to the mucous surfaces of the mouth, throat and nasal epithelium. One preferable and convenient, while highly targeted, remedy for infections related or initiating in throat is the use of a throat spray. With the right actuator, the soft mist dispersed from the throat spray will easily reach the inflamed tissue in the back of the throat to provide fast relief. Spray pattern and droplet size distribution are the most important parameters for the targeted treatment of a sore throat. Spray pattern is a term used to describe the spray angle and the shape of the plume for a fully developed spray. The droplet size is characterized once the spray is fully developed using a laser diffraction method. Fine particles (droplets with less than 10 μm mean dynamic diameter) should be as low as possible to avoid droplet deposition in the lower airways.

Other disadvantages associated with oral medicaments which aim for mucosal absorption are limitations on feasible formulations resulting in quick release and absorption, patient compliance, involuntary reflexes including nausea, salivation followed by swallowing to name a few.

The challenge is not only for the treatment of the persons infected with the SARS-CoV-2, but also for surfaces and other fomites treatment to prevent infection from being spread. It has been found in different studies that the virus sustains on surfaces for periods longer than that expected for other microbes. Thus, physical distancing from a carrier is not sufficient. Proper and regular sanitization of frequently touched surfaces is equally important. The open surfaces in public transport, restaurants, bars and other public places are potential points of infection. Other fomites include utensils, table wares etc.

Few oral sprays exist in the market. Nevertheless, in the wake of Covid19, few companies have released sprays as preventive measure. Once such spray is VIRALEZE™. It is an antiviral nasal spray for coronavirus and other viruses such as influenza and respiratory syncytial virus (RSV). VIRALEZE™ is an easy to use preventative nasal spray, which can be stored at room temperature and does not require refrigeration. VIRALEZE™ contains SPL7013, a broad spectrum antiviral, which is the active in products approved in more than 40 countries and on market in the UK, Europe, Asia, Australia and New Zealand.

Another such spray is Coldzyme, by a Swedish company Enzymatica. The mouth spray is effective in building a barrier against viruses and bacteria in the oral cavity, which includes Covid-19. The solution of Coldzyme contains water, glycerol, trypsin (Gadus morhua), ethanol (<1%), and menthol. The pH of the solution is ˜8. It is pertinent to note here that long term use of ethanol leads to stinging sensation on the skin. Also, the skin becomes dry and cracked on the long term use of ethanol. Moreover, excessive use of glycerol can lead to headaches, dizziness, bloating and nausea. Therefore, there is a need to develop natural spray based medicament which can inactivate the virus at the nasal or throat epithelium itself. The instant application is an endeavor in this direction.

All references, including any patents or patent application, cited in this specification are hereby incorporated by reference to enable full understanding of the invention. Nevertheless, such references are not to be read as constituting an admission that any of these documents form part of the common general knowledge in the art.

### OBJECT OF THE INVENTION

The main object of the present invention is to provide a fatty acid based composition for the prevention and/or treatment of viral infections.

Another object of the present invention is to provide a fatty acid based sprayable composition to prevent and/or treatment of SARS-Virus related infections.

Yet another object of the invention is to provide a composition to be delivered via oral or nasal route.

Still another object of the invention is to provide a composition used in food processing industry including but not limited to sanitizing all kinds of surfaces, packaging, table wares, eatables etc., especially where soaps and bleach based disinfectants are not recommended.

The above objects of the present invention will become apparent from the hereinafter set forth brief description of the drawings, detailed description of the invention, and claims appended herewith.

### SUMMARY OF THE INVENTION

The present invention relates to a fatty acid based composition for prevention and/or treatment of viral infections. More particularly, the present invention provides a fatty acid based sprayable composition to prevent and/or treatment of SARS-Virus related infections.

The present invention is based on the concept that enveloped viruses are protected by a fatty coating which is disrupted by detergents (usually on contact). This leads to destruction of the virus. Fatty acids that are part of every cell and present in foods, have detergent actions. Fatty acids inactivate/destroy viruses via their detergent actions. SARS CoV2 (Covid-19) is an enveloped virus that is inactivated by detergents. There is anticipated high probability that fatty acids also inactivate SARS CoV2 (Covid-19) on contact. SARS CoV2 (Covid-19) infects through nose, mouth and throat before moving to lungs.

In an embodiment, the present invention provides a composition comprising at least one fatty acid), a suitable carrier, preservative, co-solvents, and surfactants. The present composition is to be delivered via oral or nasal route.

In yet another embodiment, the present invention provides fatty acid that is selected from but not limited to the group of LA, GLA, DGLA, Oleic acid, ALA, EPA, ARA, DHA, caprylic acid, lauric acid or any other Poly-Unsaturated fatty Acid and amphipathic fatty acids or their monoglycerides.

In still another embodiment, the present invention is to provide a composition in the form of a spray, emulsion, suspension, solution, foam, ointment, liquid soap, cream, mouthwash, jelly, lozenge or pastille.

In yet another embodiment, the present invention is to provide a composition that is mucoadhesive.

Still another embodiment, the present invention provides a method of preventing and/or treating microbial infection including those caused by enveloped viruses by administering a fatty acid based composition.

Yet another embodiment of the present invention proposes a method that inhibits the growth of enveloped viruses.

In still another embodiment, the present invention provides a method that inhibits the growth of SARS-CoV-2 virus.

In yet another embodiment the present invention provides a method to disinfect the surfaces and other fomites.

In another embodiment, the present invention provides a topical administration of naturally occurring fatty acids in an optimal formulation, that may be beneficial in the prophylaxis of respiratory enveloped viruses and in particular the lipid-rich enveloped viruses such as SARS-CoV-2 virus. This could be administered by nasal and/or oral route and also as a mouthwash or linctus.

Yet another embodiment of the present invention provides use of the composition in food processing industry including but not limited to sanitizing all kinds of surfaces, packaging, table wares, eatables etc., especially where soaps and bleach based disinfectants are not recommended.

In still another embodiment, the present invention provides an antiviral composition comprising an effective amount of one or more fatty acid, a suitable carrier, a preservative, one of more cosolvents, and one or more surfactants;

wherein:
one or more free fatty acids is selected in the ratio of about 5:1 to about 1:5;
the composition comprises between 0.01% and 10% fatty acid;
the composition is a disinfectant composition comprising a pH in the range of 5-7.5; and
the antiviral activity is for enveloped virus including SARS-CoV-2.

In another embodiment, the present invention provides a composition that comprises at least one fatty acid, a suitable carrier, preservative, cosolvents, and surfactants. The proposed composition is in the form of spray. The surfactants in present fatty acid composition may be selected from polysorbate 20 (TWEEN 20), poly(ethylene glycol) 200 (PEG 200), Cyclodextrin, Triton and other suitable ether and anionic surfactants.

In yet another preferred embodiment, the present invention provides a composition wherein the at least one fatty acid is selected from the group of LA, GLA, DGLA, Oleic acid, ALA, EPA, ARA, DHA, caprylic acid, lauric acid or any other fatty acid and/or their monoglycerides.

In yet another embodiment, the present invention provides a composition wherein the composition is in the form of a spray, emulsion, suspension, solution, foam, ointment, liquid soap, cream, mouthwash, jelly, lozenge or pastille.

In yet another embodiment, the present invention provides a composition wherein the composition is in the form of nasal spray, mouth spray and topical ointment.

In yet another embodiment, the present invention provides a composition wherein the composition is mucoadhesive.

In yet another embodiment, the present invention provides a composition which is one or more of the fatty acids mixed with cyclodextrin.

### BRIEF DESCRIPTION OF THE DRAWINGS

An understanding of the antiviral composition and method of preventing and/or treating microbial infection of the present invention may be obtained by reference to the following drawings:

FIG. 1 graphically represents percentage reduction in viral load by using the composition containing LA.

FIG. 2 graphically represents percentage reduction in viral load by using the composition containing GLA.

FIG. 3 shows the particle size of ALA using Malvern zetasizer. The mean particle size obtained using the present composition was 170.4 nm when the sample was diluted 50 times.

FIG. 4 shows the particle size of GLA using Malvern zetasizer. The mean particle size obtained using the present composition was 283.2 nm when the sample was diluted 50 times.

FIG. 5 shows the particle size of LA using Malvern zetasizer. The mean particle size obtained using the present composition was 183 nm when a sample was diluted 50 times.

### DETAILED DESCRIPTION OF THE INVENTION

The details of one or more embodiments of the invention are set forth in the accompanying description below including specific details of the best mode contemplated by the inventors for carrying out the invention. The embodiments of the invention which are apparent to one skilled in the art after reading the present disclosure and on applying the common general knowledge of the technical field are within the scope of this invention.

The present invention is based on the concept that enveloped viruses are protected by a fatty coating which is disrupted by detergents (usually on contact). This leads to destruction of the virus. Fatty acids are part of every cell and in food, have detergent actions. Fatty acid can inactivate/destroy viruses via their detergent actions. SARS-CoV-2 (Covid-19) is an enveloped virus and thus there is anticipated high probability that fatty acid can also inactivate SARS-CoV-2 (Covid-19) on contact.

The present invention uses fatty acids which are long chain carboxylic acid with long aliphatic chain. No double bond in the aliphatic chain forms saturated fatty acids. If one double bond is present in the aliphatic chain, it forms monounsaturated fatty acids. Two or more double bonds in the aliphatic chain forms polyunsaturated fatty acids (PUFA).

Fatty acids, primarily Linoleic acid (LA), α-linolenic acid (ALA) and gamma-linolenic acid (GLA) are taken for the study. It was seen that each of these fatty acids showed antiviral activity when incubated with enveloped viral cells, specifically SARS-CoV-2 cells. The fatty acid used in the present composition are used in purified or extracted form. Preferably, the composition used according to the present application comprises of a single fatty acid at a level that is of other fatty acids. Specifically, it implies that the other fatty acids are not present in a single composition.

Compositions comprising fatty acids have been found by the inventors to have broad antiviral activity. This activity provides wide application for the composition. It has also been found that composition can be used in different forms which further enhances the application of the composition. Since, the SARS-Co-2 virus enters the host via the nasal epithelium, therefore it is reasonable to expect that the treatment of such mucosal surfaces with fatty acids, such as linolenic acid etc. would result in the inactivation of SARS-CoV-2, thereby retarding or inhibiting the development of COVID-19.

If infection of the nasal passages may occur or has occurred or is desired to be prevented, administration of the composition to the nasal passages may be appropriate, and the composition may be in the form of a nasal ointment or nasal spray. If infection of the throat has occurred or is desired to be prevented, the composition may further be in the form of a throat gargle, lozenge, or spray and other forms of oral and nasal delivery.

In a preferred embodiment, the present invention provides an antiviral composition comprising an effective amount of one or more free fatty acids or free fatty acids (FA or FFA), a suitable carrier, a preservative, one of more cosolvents, and one or more surfactants;

wherein,
the composition comprises between 0.01% and 10% of fatty acid;
the composition is anti-microbial preparation with a pH in the range of 5-7.5; and
the antiviral activity is for enveloped virus including SARS-CoV-2.

In another preferred embodiment, the present invention provides a composition wherein at least one fatty acid is selected from but not limited to the group of LA, GLA, DGLA, Oleic acid, ALA, EPA, ARA, DHA, caprylic acid, lauric acid or any other fatty acids.

In yet another preferred embodiment, the present invention provides a composition that is in the form of a spray, emulsion, suspension, solution, foam, ointment, liquid soap, cream, mouthwash, jelly, lozenge or pastille.

In yet another preferred embodiment, the present invention provides a composition in the form of nasal spray, mouth spray and topical ointment.

In yet another preferred embodiment, the present invention discloses that a composition is mucoadhesive.

In yet another preferred embodiment of the present invention provides a method of preventing and/or treating microbial infection including those caused by enveloped viruses by administering a fatty acid based composition.

In yet another preferred embodiment, the present invention provides a method that inhibits the growth of enveloped viruses by rupturing lipid-rich protein outer envelope coating of the virus.

In yet another preferred embodiment, the present invention provides a method that inhibits the growth of enveloped viruses.

In yet another preferred embodiment, the present invention provides a method wherein the method inhibits the growth of SARS-CoV-2 virus.

In yet another preferred embodiment of the present invention provides a method used to disinfect the surfaces and other fomites.

In yet another preferred embodiment, the present invention provides a method wherein the composition is administered topically.

In yet another preferred embodiment, the present invention provides use of the composition in food processing industry including but not limited to sanitizing all kinds of surfaces, packaging, table wares, eatables etc., especially where soaps and bleach based disinfectants are not recommended.

In yet another preferred embodiment, the present invention provides a method of treating a disease or condition caused by SARS-CoV-2, the method comprising the step of administering to a subject in need thereof a composition disclosed in above embodiments.

In yet another preferred embodiment, the present invention provides use of the composition as disclosed in above embodiments, in food processing industry including but not limited to sanitizing all kinds of surfaces, packaging, table wares, eatables etc., especially where soaps and bleach based disinfectants are not recommended.

In still another preferred embodiment, the present invention provides an antiviral composition comprising an effective amount of one or more fatty acid, a suitable carrier, a preservative, one of more cosolvents, and one or more surfactants;

Wherein,

one or more free fatty acids is selected in the ratio of about 5:1 to about 1:5;
the composition comprises between 0.01% and 10% free fatty acid;
the composition is a disinfectant composition comprising a pH in the range of 5-7.5; and
the antiviral activity is for enveloped virus including SARS-CoV-2.

In another preferred embodiment, the present invention provides a composition that comprises at least one fatty acid, a suitable carrier, preservative, cosolvents, and surfactants. The proposed composition is in the form of spray. The surfactants in present fatty acid composition may be selected from polysorbate 20 (TWEEN 20), poly(ethylene glycol) 200 (PEG 200), Cyclodextrin, Triton and other suitable ether and anionic surfactants.

In yet another preferred embodiment, the present invention provides a composition wherein the at least one fatty acid is selected from the group of LA, GLA, DGLA, Oleic acid, ALA, EPA, ARA, DHA, caprylic acid, lauric acid or any other fatty acid; and/or their monoglycerides.

In yet another preferred embodiment, the present invention provides a composition wherein the composition is in the form of a spray, emulsion, suspension, solution, foam, ointment, liquid soap, cream, mouthwash, jelly, lozenge or pastille.

In yet another preferred embodiment, the present invention provides a composition wherein the composition is in the form of nasal spray, mouth spray and topical ointment.

In yet another preferred embodiment, the present invention provides a composition wherein the composition is mucoadhesive.

In yet another preferred embodiment, the present invention provides a composition which is one or more of the fatty acids mixed with cyclodextrin.

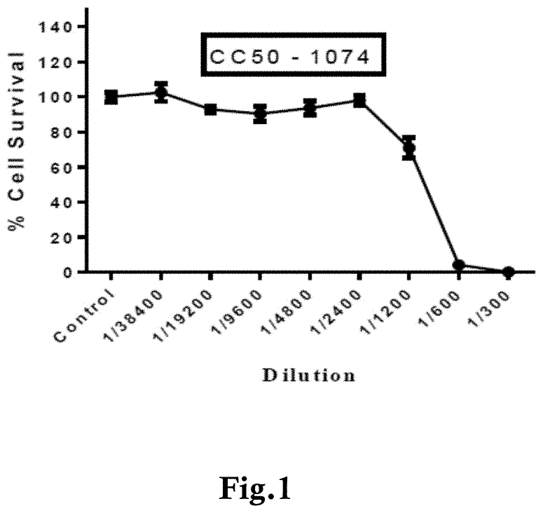

As shown in FIG. 1, graphically represents percentage reduction in viral load by using the composition containing LA.

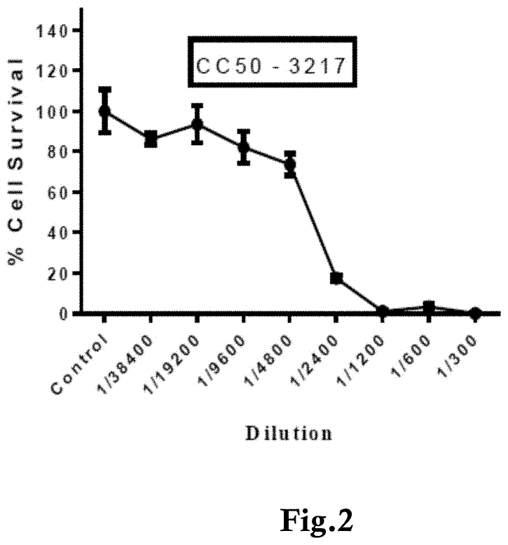

As shown in FIG. 2, graphically represents percentage reduction in viral load by using the composition containing GLA.

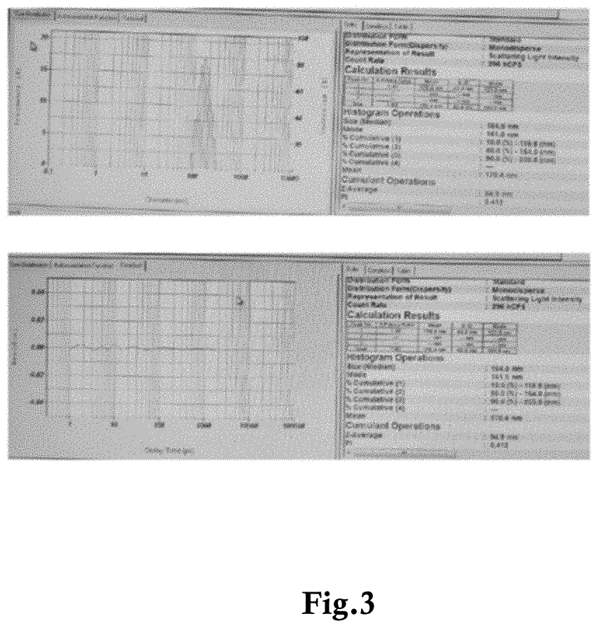

As shown in FIG. 3, the particle size of ALA using Malvern zetasizer. The mean particle size obtained using the present composition was 170.4 nm when the sample was diluted 50 times.

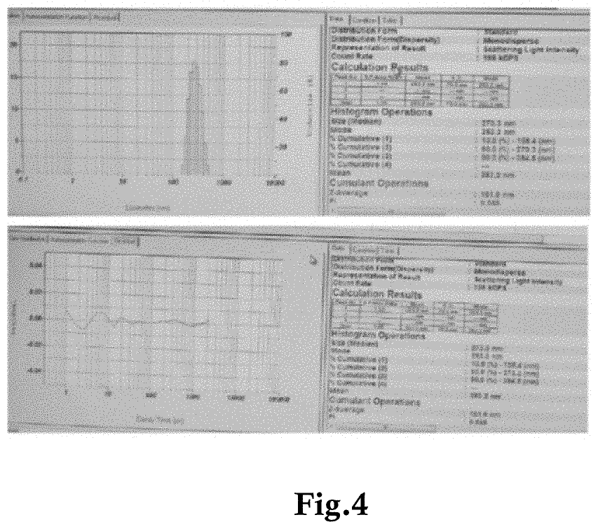

As shown in FIG. 4, the particle size of GLA using Malvern zetasizer. The mean particle size obtained using the present composition was 283.2 nm when the sample was diluted 50 times.

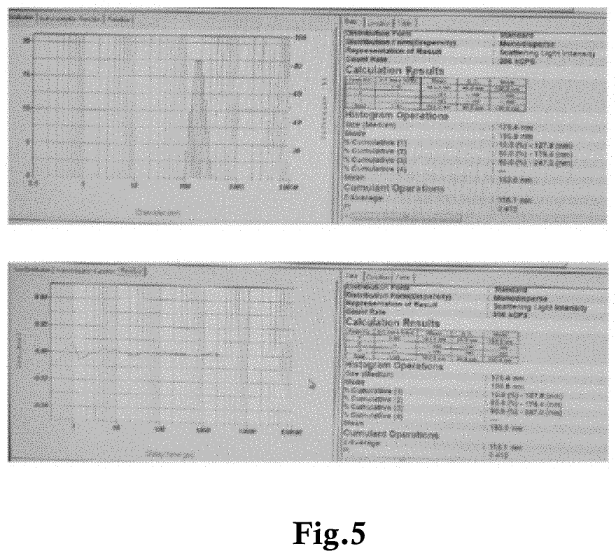

As shown in FIG. 5, the particle size of LA using Malvern zetasizer. The mean particle size obtained using the present composition was 183 nm when a sample was diluted 50 times.

### EXAMPLES

The following examples and advantages of the present invention are provided for the purpose of illustration only and are not intended to limit the scope of the present invention.

### Example 1

### Minimum Inhibitory Concentration Test for LA, ALA and GLA

Level of virus used (MOI of 1) of wild type SARS-CoV-2, WA1/2020 strain in 100 ul of medium (DMEM containing 2% FBS) with indicated concentration of the samples provided samples in a final volume of 100 ul at 37° C. for 10 min in a CO₂ incubator achieving the desired dilution of sample. Control infection samples contained virus (10 ul) alone with 90 ul of medium. Mock well contained 100 ul of medium alone. Vero E6 cells (10,000/well) are plated overnight. Spent medium to be removed and cells were washed once with the complete medium. Fresh complete medium was added to each well (75 ul) along with the sample (25 ul) from Step 6 diluted (1:4 fold as indicated in column I) to each well in triplicate making the final dilutions as indicated in column J. Vero cells were infected for one hour. Subsequently the medium was removed and cells were washed twice with complete medium. Fresh complete medium alone (100 ul) was added to each well and cells were further incubated for 72 hours. The supernatant was removed and assayed for the level of viable virus by RT-PCR assay. The highest serial dilution which achieved inhibition of the test organism was deemed to be the minimum inhibitory concentration (MIC). The following tables (1A-1E) depict the MIC of the test samples with fatty acids as well as with the synthetic detergent Triton X100 and Triolein, a non-fatty acid for comparison.

TABLE 1A
Test organism: SARS-CoV-2 in Vero6 cell line
Test sample: Triton X100 (1.71M)

| | Dilution | | | | | |
|---|---|---|---|---|---|---|
| Test | 1:1024000 | 1:512000 | 1:256000 | 1:128000 | 1:64000 | 1:32000 |
| 1 | − | − | − | − | − | + |

TABLE 1B
Test organism: SARS-CoV-2 in Vero6 cell line
Test sample: Triolein

Concentration

| Test | 6.25 mM | 12.5 mM | 25 mM | 50 mM |
|---|---|---|---|---|
| 1 | − | − | − | + |

TABLE 1C
Test organism: SARS-CoV-2 in Vero6 cell line
Test sample: LA(4.32 mM)

| | Dilution | | | | | | |
|---|---|---|---|---|---|---|---|
| Test | 1:38400 | 1:19200 | 1:9600 | 1:4800 | 1:2400 | 1:1200 | 1:600 |
| 1 | − | − | − | − | − | − | + |

TABLE 1D
Test organism: SARS-CoV-2 in Vero6 cell line
Test sample: ALA (11.78 mM)

Dilution

| Test | 1:4800 | 1:2400 | 1:1200 | 1:600 |
|---|---|---|---|---|
| 1 | − | − | − | + |

TABLE 1E
Test organism: SARS-CoV-2 in Vero6 cell line
Test sample: GLA (2.96 mM)

| | Dilution | | | | | |
|---|---|---|---|---|---|---|
| Test | 1:76800 | 1:38400 | 1:19200 | 1:9600 | 1:4800 | 1:2400 |
| 1 | − | − | − | − | + | + |

### Example 2

### Antiviral Activities of LA, ALA and GLA

Antiviral activity of the given composition was evaluated using vero cells and SARS-CoV-2 virus (WA1/2020) strain. First, virus was incubated for 10 minutes with suggested concentration of fatty acids (see table 2A) along with the mock control (virus only). These mixtures were diluted four times to obtain the non-toxic dilution and equal amount of these were added to the seeded vero cells. These infected cells were then incubated for 48/72 hours and subsequently the supernatant was collected. This supernatant was subjected to virus measurement using plaque assay. The results are represented in terms of percentage viral reduction in the sample in Table 2B below. The graphical representation of antiviral activity of LA and GLA is shown in FIG. 1 and FIG. 2 respectively.

TABLE 2A

| | Conc./dilution used | |
|---|---|---|
| | for pre-incubation | Conc./dilution |
| Sample | with virus | added to cells |
| Virus only | NA | NA |
| Triton X | 50 μM | 1:12800 |
| Triolein | 200 mM | 50 mM |
| LA | 1:600(4.317 mM) | 1:2400 |
| GLA | 1:12000(2.9632 mM) | 1:4800 |
| ALA | 1:300 (11.78 mM) | 1:4800 |

TABLE 2B

| Sample | % viral reduction |
|---|---|
| Virus only | NA (0) |
| Triton X100 | 95.23 |
| Triolein | 2.21 |
| ALA | 97.13 |
| LA | 97.42 |
| GLA (1:1200) | 96.98 |

The above results clearly show that the activities of LA, ALA and GLA can be very well compared to the synthetic detergent Triton X. The experiment clearly shows that these fatty acids have the ability to reduce viral load.

### Example 3

### Compositions Comprising LA, ALA and GLA with their Particle Size

The present composition was standardized to check for the most stable composition and for their particle size. The control was first run with a blank trial, i.e. without the use of fatty acid. Then various concentrations of the composition were standardized with different percentage of the composition and the batches were prepared using different processes such as magnetic stirring and by HSH of IKA at different centrifugal speed. It was seen that magnetic stirring results in the best of batches for the requisite composition.

The resultant batches were then checked for various parameters such as:
a) Microscopy
b) Particle size analysis
c) Phase separation

Following tables depict the compositions of different preparation sample for the preparation of the nasal/mouth spray.

A) Fatty Acids ALA, GLA and LA for Particle Size and Stability of Composition ALA

Composition

TABLE 3A

| S. No. | Ingredients | Quantity |
|---|---|---|
| 1. | ALA (ml) | 1.5 |
| 2. | Xantham gum (mg) | 10 |
| 3. | Guar gum (mg) | 10 |
| 4. | Tween 80 (ml) | 0.5 |
| 5. | Span 80 (ml) | 0.5 |
| 6. | Benzalkonium chloride (ml) | 0.016 |
| 7. | Water (ml) | 8 |

Particle Size Analysis

Particle size was analyzed using Malvern zetasizer. Results are displayed in the table below and in FIG. 3.

TABLE 3B

| S. No. | Dilution | Mean size | pI |
|---|---|---|---|
| 1. | 50 | 170.4 | 0.412 |

GLA

Composition

TABLE 3C

| S. No. | Ingredients | Quantity |
|---|---|---|
| 1. | GLA (ml) | 1.5 |
| 2. | Xanthan gum (mg) | 10 |
| 3. | Guar gum (mg) | 10 |
| 4. | Tween 80 (ml) | 0.6 |
| 5. | Span 80 (ml) | 0.6 |
| 6. | Benzalkonium chloride (ml) | 0.016 |
| 7. | Water (ml) | 10 |

Particle Size Analysis

Particle size was analyzed using Malvern zetasizer. Results are displayed in the table below and in FIG. 4.

TABLE 3D

| S. no. | Dilution | Mean size | pI |
|---|---|---|---|
| 1. | 50 | 283.2 | 0.565 |

LA

Composition

TABLE 3E

| S. No. | Ingredients | Quantity |
|---|---|---|
| 1. | LA (ml) | 1.5 |
| 2. | Xantham gum (mg) | 10 |
| 3. | Guar gum (mg) | 10 |
| 4. | Tween 80 (ml) | 0.6 |
| 5. | Span 80 (ml) | 0.6 |
| 6. | Benzalkonium chloride (ml) | 0.016 |
| 7. | Water (ml) | 8 |

Particle Size Analysis

Particle size was analyzed using Malvern zetasizer. Results are displayed in the table below and in FIG. 5.

TABLE 3F

| S. No. | Dilution | Mean size | pI |
|---|---|---|---|
| 1. | 50 | 183.0 | 0.412 |

It was observed that the above compositions were stable for more than 100 hours at room temperature

Cyclodextrin

Composition

TABLE 3G

| S. No. | Ingredients | Quantity |
|---|---|---|
| 1. | ALA (ml) | 1.5 |
| 2. | Xantham gum (mg) | 10 |
| 3. | Guar gum (mg) | 10 |
| 4. | Cyclodextrin (ml) | 0.6 |
| 5. | Span 80 (ml) | 0.6 |
| 6. | Benzalkonium chloride (ml) | 0.016 |
| 7. | Water (ml) | 8 |

Many modifications and other embodiments of the invention set forth herein will readily occur to one skilled in the art to which the invention pertain having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. An antiviral composition comprising an effective amount of one or more fatty acid a suitable carrier, a preservative, one of more cosolvents, and one or more surfactants;
wherein,
the composition comprises between 0.01% and 10% fatty acid;
the composition is a disinfectant composition comprising a pH in the range of 5-7.5; and
the antiviral activity is for enveloped virus including SARS-CoV-2.

2. The composition as claimed in claim 1, wherein the at least one fatty acid is selected from the group of LA, GLA, DGLA, Oleic acid, ALA, EPA, ARA, DHA, caprylic acid, lauric acid or any other Poly-Unsaturated fatty acid and amphipathic fatty acids.

3. The composition as claimed in claim 1, wherein the composition is in the form of a spray, emulsion, suspension, solution, foam, ointment, liquid soap, cream, mouthwash, jelly, lozenge or pastille.

4. The composition as claimed in claim 1, wherein the composition is in the form of nasal spray, mouth spray and topical ointment.

5. The composition as claimed in claim 1, wherein the composition is mucoadhesive.

6. A method of preventing and/or treating microbial infection including those caused by enveloped viruses by administering a fatty acid based composition.

7. The method as claimed in claim 6, wherein the method inhibits the growth of enveloped viruses by rupturing lipid-rich protein outer envelope coating of the virus.

8. The method as claimed in claim 7, wherein the method inhibits the growth of SARS-CoV-2 virus.

9. The method as claimed in claim 7, wherein the method is used to disinfect the surfaces and other fomites.

10. The method as claimed in claim 7, wherein the composition is administered topically.

11. An antiviral composition comprising an effective amount of one or more fatty acid, a suitable carrier, a preservative, one of more cosolvents, and one or more surfactants;
wherein,
one or more free fatty acids is selected in the ratio of about 5:1 to about 1:5;
the composition comprises between 0.01% and 10% free fatty acid;
the composition is a disinfectant composition comprising a pH in the range of 5-7.5; and
the antiviral activity is for enveloped virus including SARS-CoV-2.

12. The composition as claimed in claim 11, wherein the at least one fatty acid is selected from the group of LA, GLA, DGLA, Oleic acid, ALA, EPA, ARA, DHA, caprylic acid, lauric acid or any other fatty acid; and/or their monoglycerides.

13. The composition as claimed in claim 11, wherein the composition is in the form of a spray, emulsion, suspension, solution, foam, ointment, liquid soap, cream, mouthwash, jelly, lozenge or pastille.

14. The composition as claimed in claim 11, wherein the composition is in the form of nasal spray, mouth spray and topical ointment.

15. The composition as claimed in claim 11, wherein the composition is mucoadhesive.

16. The composition as claimed in claim 11, wherein the fatty acids mixed with cyclodextrin as a preferred surfactant.

17. Use of the composition as claimed in claim 11, in food processing industry including but not limited to sanitizing all kinds of surfaces, packaging, table wares, eatables etc., especially where soaps and bleach based disinfectants are not recommended.
